(19) United States
(12) Patent Application Publication
(10) Pub. No.: US 2014/0024552 A1
(43) Pub. Date: Jan. 23, 2014

(54) COMPOSITIONS AND METHODS FOR DIAGNOSING OVARIAN CANCER

(71) Applicants: Zhen Zhang, Dayton, MD (US); Daniel W. Chan, Clarksville, MD (US)

(72) Inventors: Zhen Zhang, Dayton, MD (US); Daniel W. Chan, Clarksville, MD (US)

(73) Assignee: THE JOHNS HOPKINS UNIVERSITY, Baltimore, MD (US)

(21) Appl. No.: 13/985,140

(22) PCT Filed: Feb. 15, 2012
(86) PCT No.: PCT/US2012/025255

§ 371 Date: Oct. 7, 2013

Related U.S. Application Data
(60) Provisional application No. 61/443,053, filed on Feb. 15, 2011.

(51) Int. Cl. G01N 33/574

(52) U.S. Cl. CPC G01N 33/57449
USPC 506/9; 506/18; 506/16

(57) ABSTRACT

The invention provides methods and compositions for distinguishing ovarian cancer from a benign pelvic mass using two or more of the following biomarkers: IL-6, MMP9, tPA, IGFBP2, MMP7, Tenascin, NAP2, glycodelin, MCSF, MMP2, Inhibin A, uPAR, and EGFR. The methods are useful in distinguishing a benign pelvic mass from ovarian cancer in subjects, particularly in subjects identified as having increased CA125 levels.

### CROSS-REFERENCE TO RELATED APPLICATION

This application claims the benefit of U.S. Provisional Application No. 61/443,053, filed Feb. 15, 2011, the entire contents of which are incorporated herein by reference.

### BACKGROUND OF THE INVENTION

Ovarian cancer is one of the most deadly cancers among women. Currently the majority of ovarian cancer patients are diagnosed at late stages resulting in an extremely poor prognosis for such subjects. The ability to distinguish malignant growths from benign ovarian masses, prior to surgery, is urgently required to ensure that women receive appropriate therapy as soon as possible.

### SUMMARY OF THE INVENTION

As described below, the present invention features compositions and methods for diagnosing ovarian cancer. In particular embodiments, the invention provides methods from distinguishing ovarian cancer from a benign pelvic mass using one or more of the following biomarkers: IL-6, MMP9, tPA, IGFBP2, MMP7, Tenascin, NAP2, glycodelin, MCSF, MMP2, Inhibin A, uPAR, and EGFR. The methods are useful in distinguishing a benign pelvic mass from ovarian cancer in subjects, particularly in subjects identified as having increased CA125 levels.

In one aspect, the invention generally features methods for identifying ovarian cancer in a subject, the method involving identifying an increased level of CA125 and one or more (e.g., two, three, four, five, six, seven, eight, nine, ten, eleven or all) of the following Marker nucleic acid molecules or polypeptides: IL-6, MMP9, tPA, IGFBP2, MMP7, Tenascin, NAP2, Glycodelin, MCSF, MMP2, InhibinA, uPAR, and EGFR in a biological sample derived from the subject, relative to the level present in a reference, thereby identifying ovarian cancer in the subject.

In another aspect, the invention features methods for distinguishing ovarian cancer from a benign pelvic mass in a subject, the method involves measuring the level of CA125 and one or more (e.g., two, three, four, five, six, seven, eight, nine, ten, eleven or all) of the following Marker nucleic acid molecules or polypeptides: MMP9, tPA, IGFBP2, MMP7, Tenascin, NAP2, Glycodelin, MCSF, MMP2, InhibinA, uPAR, and EGFR in a biological sample derived from the subject, where an alteration in the level relative to the level present in a reference, identifies ovarian cancer in the subject, and failure to identify an increase in the levels identifies the subject as having a benign pelvic mass. In another aspect, the invention features methods for identifying ovarian cancer in a subject, the method comprising identifying a subject as having an increased level of a CA125 or HE4 in serum of the subject, and detecting an altered level of a Marker nucleic acid molecule or polypeptide selected from the group consisting of MMP9, tPA, IGFBP2, MMP7, Tenascin, NAP2, Glycodelin, MCSF, MMP2, InhibinA, uPAR, and EGFR in a biological sample derived from the subject, relative to the level present in a reference, thereby identifying ovarian cancer in the subject.

In another aspect, the invention features methods for distinguishing ovarian cancer from a benign pelvic mass in a subject, the method comprising identifying a subject as having an increased level of a CA125 or HE4 in a biological sample of the subject, and measuring the level of one or more Marker nucleic acid molecules or polypeptides selected from the group consisting of MMP9, tPA, IGFBP2, MMP7, Tenascin, NAP2, Glycodelin, MCSF, MMP2, InhibinA, uPAR, and EGFR in a biological sample derived from the subject, where an alteration in the Marker level relative to the level present in a reference, identifies ovarian cancer in the subject, and failure to identify an increase in the levels identifies the subject as having a benign pelvic mass.

In another aspect, the invention features methods for determining the Marker profile of ovarian cancer, the method comprising characterizing the level of two or more of the following Markers: MMP9, tPA, IGFBP2, MMP7, Tenascin, NAP2, Glycodelin, MCSF, MMP2, InhibinA, uPAR, and EGFR in a biologic sample, where the level of Marker in the sample relative to the level in a reference determines the Marker profile of the ovarian cancer.

In another aspect, the invention features kits for identifying ovarian cancer in a biological sample, the kit comprising at least one polynucleotide molecule or capture molecule (e.g., antibody, aptamer) capable of specifically binding or hybridizing to a MMP9, tPA, IGFBP2, MMP7, Tenascin, NAP2, Glycodelin, MCSF, MMP2, InhibinA, uPAR, or EGFR polypeptide or nucleic acid molecule, and directions for using the kit for the diagnosis of ovarian cancer according to any methods delineated herein. In one embodiment, antibody binding is detected by fluorescence, by autoradiography, by an immunoassay, by an enzymatic assay, or by a colorimetric assay.

In another aspect, the invention features microarrays containing at least two nucleic acid molecules, or fragments thereof, bound to a solid support, where the two nucleic acid molecules hybridize to a nucleic acid molecule selected from the group consisting of MMP9, tPA, IGFBP2, MMP7, Tenascin, NAP2, Glycodelin, MCSF, MMP2, InhibinA, uPAR, and EGFR.

In another aspect, the invention features microarrays containing at least polypeptides, or fragments thereof, bound to a solid support, where the two nucleic acid molecules hybridize to a nucleic acid molecule selected from the group consisting of MMP9, tPA, IGFBP2, MMP7, Tenascin, NAP2, Glycodelin, MCSF, MMP2, InhibinA, uPAR, and EGFR.

In another aspect, the invention features microarrays containing at least two antibodies, or fragments thereof, bound to a solid support, where the antibodies specifically bind to a polypeptide two or more polypeptides selected from the group consisting of MMP9, tPA, IGFBP2, MMP7, Tenascin, NAP2, Glycodelin, MCSF, MMP2, InhibinA, uPAR, and EGFR.

In another aspect, the invention features panels of markers for characterizing ovarian cancer or a bening pelvic mass, the markers comprising the following combinations: MMP9, tPA, IGFBP2, MMP7, Tenascin, NAP2, Glycodelin, MCSF, MMP2, InhibinA, uPAR, and EGFR; IGFBP2, MMP7, and tPA; HE4, Transthyretin, Apo A-1, beta 2-Microglobulin, Transferrin and Cancer Antigen 125; IGFBP2, MMP7, and tPA; IGFBP2, MMP7 and CA125; IGFBP2 and MMP7; MMP9, tPA, IGFBP2, MMP7 Tenascin NAP2, glycodelin, MCSF, MMP2, Inhibin A, uPAR, EGFR, Transthyretin, Apo A-1, beta 2-Microglobulin, Transferrin and Cancer Antigen 125; IGFBP2, MMP7 and CA125; IGFBP2 and MMP7; MMP9, tPA, IGFBP2, MMP7 Tenascin NAP2, glycodelin, MCSF, MMP2, Inhibin A, uPAR, EGFR, Transthyretin. Apo A-1, beta 2-Microglobulin, Transferrin, Cancer Antigen 125, and HE4. In various embodiments of the above aspects of the invention or any other aspect of the invention delineated herein, a combination of the invention further includes IL-6 as an ovarian cancer marker. Levels of IL-6 are altered relative to a control in ovarian cancer. In still other embodiments of the above aspects, the biological sample is an ovarian tissue sample, tumor sample, needle biopsy, blood, blood serum, plasma, ascites, pleural effusion, or urine. In still other embodiments of the above aspects, the Marker nucleic acid molecule or polypeptide are MMP7, Tenascin C, NAP2, uPAR, and MMP9. In still other embodiments of the above aspects, the Markers are MMP7, Tenascin C and NAP2. In still other embodiments of the above aspects, the Markers are IGFBP2 and MMP7. In still other embodiments of the above aspects, the Markers are IGFBP2, MMP7 and CA125. In still other embodiments of the above aspects, the Markers are HE4, Transthyretin, Apolipoprotein A-1, beta 2-Microglobulin, Transferrin and Cancer Antigen 125. In still other embodiments of the above aspects, the Markers are IGFBP2, MMP7, and tPA. In still other embodiments of the above aspects, the Markers further comprise IL-6. In still other embodiments of the above aspects, the reference is the level of a marker present in a corresponding biological sample derived from a healthy subject In still other embodiments of the above aspects, the method further involves quantifying the level of Transthyretin (TT or prealbumin), Apolipoprotein A-1 (Apo A-1), beta 2-Microglobulin (beta 2M), Transferrin (Tfr) and Cancer Antigen 125 (CA 125 II) and/or HE4 in a biological sample derived from the subject. In still other embodiments of the above aspects, the Marker is measured in an immunoassay, radioassay, hybridization assay, mass spectrometry assay, or a multiplexed assay. In still other embodiments of the above aspects, the immunoassay is an ELISA.

Particular combinations useful in the methods and compositions of the invention include the following:

MMP9, tPA, IGFBP2, MMP7, Tenascin, NAP2, Glycodelin, MCSF, MMP2, InhibinA, uPAR, and EGFR. MMP9, tPA, IGFBP2, MMP7, Tenascin, NAP2, Glycodelin, MCSF, MMP2, InhibinA, uPAR, EGFR, Transthyretin (TT or prealbumin) Apolipoprotein A-1 (Apo A-1), beta 2-Microglobulin (beta 2M), Transferrin and Cancer Antigen 125 (CA 125 II)

MMP9, tPA, IGFBP2, MMP7, Tenascin, NAP2, Glycodelin, MCSF, MMP2, InhibinA, uPAR, EGFR, Transthyretin, Apo A-1, beta 2-Microglobulin, Transferrin, CA 125, and HE4;

MMP7, Tenascin C, NAP2, uPAR, and MMP9;

CA125, MMP7, Tenascin C, and NAP2;

MMP7, Tenascin C, and NAP2;

CA125, MMP7, NAP2, and IGFBP2;

MMP7, MMP9, NAP2, and IGFBP2;

CA125, IGFBP2, and MMP7

HE4 and Transthyretin, Apo A-1, beta 2-Microglobulin, Transferrin, CA 125

IGFBP2 is useful in each of the following combinations: IGFBP2, MMP7, tPA, MMP9, and NAP2; IGFBP2 and MMP9; IGFBP2 and tPA, IGFBP2 and MMP7, IGFBP2 and Tenascin C, IGFBP2 and NAP2; IGFBP2 and Glycodelin, IGFBP2 and MCSF; IGFBP2 and MMP2; IGFBP2 and InhibinA; IGFBP2 and uPAR, and IGFBP2 and EGFR. IGFBP2 and CA125; IGFBP2, CA125 and HE4. IGFBP2, Transthyretin, Apo A-1, beta 2-Microglobulin, Transferrin, and CA 125 IGFBP2, Transthyretin, Apo A-1, beta 2-Microglobulin, Transferrin, CA 125, and HE4.

In particular embodiments, the following combinations are useful in the methods of the invention: MMP9, tPA, IGFBP2, MMP7, Tenascin, NAP2, Glycodelin, MCSF, MMP2, InhibinA, and uPAR; IGFBP2 and MMP7; IGFBP2, MMP7, and tPA.

The invention provides compositions and methods for diagnosing ovarian cancer. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

By “biologic sample” is meant any tissue, cell, fluid, or other material derived from an organism. For example, tissue samples include cell samples and biopsy samples. Bodily fluids include but are not limited to, blood, blood serum, plasma, saliva, urine, peritoneal fluid, ascites, pleural effusions, and ovarian cyst fluid.

By “capture molecule” is meant any polypeptide or polynucleotide capable of specifically binding a polypeptide of interest.

By “reference” is meant a standard of comparison. For example, the MMP9, tPA, IGFBP2, MMP7, Tenascin, NAP2, Glycodelin, MCSF, MMP2, InhibinA, uPAR, and/or EGFR polypeptide or polynucleotide level present in a patient sample may be compared to the level of said polypeptide or polynucleotide present in a corresponding healthy cell or tissue or in a neoplastic cell or tissue that lacks a propensity to metastasize.

By “periodic” is meant at regular intervals. Periodic patient monitoring includes, for example, a schedule of tests that are administered daily, bi-weekly, bi-monthly, monthly, bi-annually, or annually.

By “marker” is meant any protein or polynucleotide having an alteration in expression level or activity that is associated with a disease or disorder.

By “Marker profile” is meant a characterization of the expression or expression level of two or more polypeptides or polynucleotides. In particular, the levels of one or more of the following markers: MMP9, tPA, IGFBP2, MMP7, Tenascin, NAP2, Glycodelin, MCSF, MMP2, InhibinA, uPAR, EGFR, Transthyretin (TT or prealbumin), Apolipoprotein A-1 (Apo A-1), beta 2-Microglobulin (beta 2M), Transferrin (Tfr), Cancer Antigen 125 (CA 125 11) and/or HE4.

By “matrix metalloproteinase 9 (MMP9) polypeptide” is meant a polypeptide having at least 85% sequence identity to UniProtKB/Swiss-Prot Ref No. P14780.

By “MMP9 polynucleotide” is meant a polynucleotide encoding an MMP9 polypeptide.

By “tissue plasminogen activator (tPA) is meant a polypeptide having at least 85% sequence identity to UniProtKB/Swiss-Prot Ref No. P00750. By “tPA polynucleotide” is meant a nucleic acid molecule encoding an tPA polypeptide.

By “insulin-like growth factor-binding protein 2 (IGFBP2) polypeptide” is meant a polypeptide having at least 85% sequence identity to UniProtKB/Swiss-Prot Ref No. P18065.

By “insulin-like growth factor-binding protein 2 (IGFBP2) polynucleotide” is meant a nucleic acid molecule encoding an IGFBP2 polypeptide.

By “matrix metalloproteinase-7 (MMP7) polypeptide” also termed matrilysin is meant a polypeptide having at least 85% sequence identity to UniProtKB/Swiss-Prot Ref No. P09237.

By “MMP7 polynucleotide” is meant a nucleic acid molecule encoding an MMP7 protein.

By “tenascin polypeptide” is meant a polypeptide having at least 85% sequence identity to UniProtKB/Swiss-Prot Ref No. P24821.

By “tenascin polynucleotide” is meant a nucleic acid molecule encoding a tenascin polypeptide.

By “nucleosome assembly protein 2 (NAP2) polypeptide” also termed nucleosome assembly protein 1-like 4 (NAP1L4) is meant a protein having at least 85% sequence identity to UniProtKB/Swiss-Prot Ref No. Q99733.

By “NAP2 polynucleotide” meant a nucleic acid molecule encoding a NAP2 protein.

By “glycodelin polypeptide” is meant a protein having at least 85% sequence identity to UniProtKB/Swiss-Prot Ref No. P09466.

By “glycodelin polynucleotide” is meant a nucleic acid molecule encoding a glycodelin protein.

By “macrophage-specific colony-stimulating factor 1 (MCSF) is meant a protein having at least 85% sequence identity to UniProtKB/Swiss-Prot Ref No P09603.

By “MCSF polynucleotide” is meant a nucleic acid molecule encoding a MCSF protein.

By “matrix metalloproteinase-2 (MMP2) polypeptide” is meant a protein having at least 85% sequence identity to UniProtKB/Swiss-Prot Ref No P08253.

By “MMP2 polynucleotide” is meant a nucleic acid molecule encoding a MMP2 protein.

By “InhibinA polypeptide” is meant a protein having at least 85% sequence identity to UniProtKB/Swiss-Prot Ref No. P05111.

By “InhibinA polynucleotide” is meant a nucleic acid molecule encoding a inhibinA protein.

By “urokinase-type plasminogen activator receptor (uPAR) polypeptide” is meant a protein having at least 85% sequence identity to UniProtKB/Swiss-Prot Ref No Q03405. By “uPAR polynucleotide” is meant a nucleic acid molecule encoding a uPAR protein.

By “epidermal growth factor receptor (EGFR) polypeptide” is meant a protein having at least 85% sequence identity to UniProtKB/Swiss-Prot Ref No P00533.

By “EGFR polynucleotide” is meant a nucleic acid molecule encoding a EGFR protein.

By “increased level of CA125” is meant greater than about 35 international units or greater than the amount present in a reference. Typically, levels of CA125 that are less than 35 IU are not associated with ovarian cancer.

By “antibody” is meant any immunoglobulin polypeptide, or fragment thereof, having immunogen binding ability.

“Detect” refers to identifying the presence, absence or amount of the object to be detected.

By “immunological assay” is meant an assay that relies on an immunological reaction, for example, antibody binding to an antigen. Examples of immunological assays include ELISAs, Western blots, immunoprecipitations, and other assays known to the skilled artisan.

“Microarray” means a collection of nucleic acid molecules or polypeptides from one or more organisms arranged on a solid support (for example, a chip, plate, or bead). These nucleic acid molecules or polypeptides may be arranged in a grid where the location of each nucleic acid molecule or polypeptide remains fixed to aid in identification of the individual nucleic acid molecules or polypeptides.

By “multiplex assay” is meant an assay where two or more analystes are detected concurrently.

By “panel” is meant a collection of molecules. If desired, the panel is fixed to a solid subtrate.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides an analysis of the use of the following biomarkers: MMP9, tPA, IGFBP2, MMP7 Tenascin NAP2, glycodelin, MCSF, MMP2, Inhibin A, uPAR, and EGFR in discriminating between ovarian cancer and a benign pelvic mass.

FIG. 2 provides the human amino acid sequences of MMP9, tPA, IGFBP2, MMP7, Tenascin, NAP2, Glycodelin, MCSF, MMP2, InhibinA, uPAR, and EGFR.

FIG. 3 provides an analysis of the use of IGFBP2 and MMP7 in discriminating between ovarian cancer and a benign pelvic mass.

FIG. 4 provides an analysis of the use of the following biomarkers: IGFBP2, MMP7 and CA125 in discriminating between ovarian cancer and a benign pelvic mass.

FIG. 5 provides an analysis of the use of the following biomarkers: HE4 and markers of the commercially available test Oval (i.e., Transthyretin (TT or prealbumin) Apolipoprotein A-1 (Apo A-1), beta 2-Microglobulin (beta 2M), Transferrin and Cancer Antigen 125 (CA 125 II)) in discriminating between ovarian cancer and a benign pelvic mass.

FIG. 6 provides an analysis of IGFBP2 and markers of the commercially available test Oval test in discriminating between ovarian cancer and a benign pelvic mass.

FIG. 7 provides an analysis of the use of the following biomarkers: IGFBP2, MMP7, and tPA in discriminating between ovarian cancer and a benign pelvic mass.

### DETAILED DESCRIPTION OF THE INVENTION

The invention features compositions and methods that are useful for diagnosing ovarian cancer.

The invention is based, at least in part, on the discovery of a panel of biomarkers that provides a high level of specificity in identifying women with benign pelvic masses while maintaining a high level of sensitivity in detecting ovarian cancer. This panel of biomarkers includes one or more of the following markers: Interleukin 6, MMP9, tPA, IGFBP2, MMP7, Tenascin, NAP2, Glycodelin, MCSF, MMP2, InhibinA, uPAR, EGFR whose levels are altered in ovarian cancer. In particular, MMP9 is increased; tPA is increased; IGFBP2 is increased; MMP7 is increased; Tenascin C is increased; NAP2 is reduced; Glycodelin is increased; MCSF is increased; MMP2 is increased; InhibinA is increased; uPAR is increased; and EGFR is decreased. In certain embodiments, use of a combination of these markers identifies ovarian cancer. In other embodiments, the invention provides methods of using one or more of these markers to distinguish ovarian cancer from a benign pelvic mass in subject's having high levels of CA125.

In other embodiments, MMP9, tPA, IGFBP2, MMP7, Tenascin, NAP2, Glycodelin, MCSF, MMP2, InhibinA, uPAR, EGFR, and Interleukin-6 (IL-6) are used alone or in combination with any one or more of the following biomarkers whose levels are altered in ovarian cancer: Transthyretin (TT or prealbumin) is reduced in cancer, Apolipoprotein A-1 (Apo A-1) is reduced in cancer, beta 2-Microglobulin (beta 2M) is increased in cancer, Transferrin (Tfr) is reduced in cancer, Cancer Antigen 125 (CA 125 11) is increased and/or HE4 is increased.

### Diagnostics

The present invention features diagnostic assays for detecting ovarian cancer or for pre-operatively distinguishing a benign pelvic mass from ovarian cancer, particularly in a subject identified as at an increased risk of having ovarian cancer due to altered levels of Transthyretin (TT or prealbumin), Apolipoprotein A-1 (Apo A-1), beta 2-Microglobulin (beta 2M), Transferrin (Tfr), Cancer Antigen 125 (CA 125 II) and/or HE4 in a biological sample of the subject. In one embodiment, levels of any one or more of the following markers IL-6, MMP9, tPA, IGFBP2, MMP7, Tenascin, NAP2, Glycodelin, MCSF, MMP2, InhibinA, uPAR, EGFR, Transthyretin (TT or prealbumin), Apolipoprotein A-1 (Apo A-1), beta 2-Microglobulin (beta 2M), Transferrin (Tfr) and Cancer Antigen 125 (CA 125 II) and/or HE4 are measured in a biological sample of the subject, and used to pre-operatively characterize a pelvic mass as likely to be benign or cancerous.

Standard methods may be used to measure levels of a marker in any biological sample. Biological samples include tissue samples (e.g., cell samples, biopsy samples) and bodily fluids, including, but not limited to, blood, blood serum, plasma, saliva, urine, peritoneal fluid and ovarian cyst fluid, ascites, and pleural effusions. Exemplary methods for measuring altered levels of polypeptides include immunoassay, ELISA, western blotting and radioimmunoassay or other assays described herein. Altered levels of MMP9, tPA, IGFBP2, MMP7, Tenascin, NAP2, Glycodelin, MCSF, MMP2, InhibinA, uPAR, EGFR alone or in combination with one or more additional markers, such as Transthyretin (TT or prealbumin), Apolipoprotein A-1 (Apo A-1), beta 2-Microglobulin (beta 2M), Transferrin (Tfr) and Cancer Antigen 125 (CA 125 11) and/or HE4, are considered as indicative of ovarian cancer. The alteration in MMP9, tPA, IGFBP2, MMP7, Tenascin, NAP2, Glycodelin, MCSF, MMP2, InhibinA, uPAR, EGFR levels may be by at least about 10%, 25%, 50%, 75% or more. In one embodiment, any alteration in the level of one or more markers of the invention relative to a control is indicative of ovarian cancer. In another embodiment, altered levels of MMP9, tPA, IGFBP2, MMP7, Tenascin, NAP2, Glycodelin, MCSF, MMP2, InhibinA, uPAR, and/or EGFR, in combination with Transthyretin (TT or prealbumin), Apolipoprotein A-1 (Apo A1), beta 2-Microglobulin (beta 2M), Transferrin (Tfr), Cancer Antigen 125 (CA 125 II) and/or HE4 are used to distinguish ovarian cancer from a benign pelvic mass prior to surgery. Suitable controls indicate the levels present in a sample obtained from a healthy control subject.

Any suitable method can be used to detect one or more of the markers described herein. Successful practice of the invention can be achieved with one or a combination of methods that can detect and, if desired, quantify the markers. These methods include, without limitation, hybridization-based methods, including those employed in biochip arrays, mass spectrometry (e.g., laser desorption/ionization mass spectrometry), fluorescence (e.g. sandwich immunoassay), surface plasmon resonance, ellipsometry and atomic force microscopy. Expression levels of markers (e.g., polynucleotides or polypeptides) are compared by procedures well known in the art, such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays. Methods may further include one or more of electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)ⁿ, atmospheric pressure photoionization mass spectrometry (APPI-MS), APPI-MS/MS, and APPI-(MS)ₙ, quadrupole mass spectrometry, fourier transform mass spectrometry (FTMS), and ion trap mass spectrometry, where n is an integer greater than zero.

In particular embodiment, multiple markers selected from MMP9, tPA, IGFBP2, MMP7, Tenascin, NAP2, Glycodelin, MCSF, MMP2, InhibinA, uPAR, EGFR, Transthyretin (TT or prealbumin), Apolipoprotein A-1 (Apo A-1), beta 2-Microglobulin (beta 2M), Transferrin (Tfr), Cancer Antigen 125 (CA 125 II) and/or HE4 are measured. The use of multiple markers increases the predictive value of the test and provides greater utility in diagnosis, toxicology, patient stratification and patient monitoring. The process called “Pattern recognition” detects the patterns formed by multiple markers and greatly improves the sensitivity and specificity of clinical proteomics for predictive medicine. Subtle variations in data from clinical samples indicate that certain patterns of protein expression can predict phenotypes such as the presence or absence of a certain disease, a particular stage of cancer progression, or a positive or adverse response to drug treatments.

Expression levels of particular nucleic acids or polypeptides are correlated with ovarian cancer, and thus are useful in diagnosis. Antibodies that bind a polypeptide described herein, oligonucleotides or longer fragments derived from a nucleic acid molecule encoding such polypeptides, or any other method known in the art may be used to monitor expression of a polynucleotide or polypeptide of interest (.g., MMP9, tPA, IGFBP2, MMP7, Tenascin, NAP2, Glycodelin, MCSF, MMP2, InhibinA, uPAR, EGFR, Transthyretin (IT or prealbumin), Apolipoprotein A-1 (Apo A-1), beta 2-Microglobulin (beta 2M), Transferrin (Tfr) and Cancer Antigen 125 (CA 125 II) and/or HE4). Detection of an alteration relative to a normal, reference sample can be used as a diagnostic indicator of ovarian cancer. In particular embodiments, the expression of a MMP9, tPA, IGFBP2, MMP7, Tenascin, NAP2, Glycodelin, MCSF, MMP2, InhibinA, uPAR, and/or EGFR polypeptide is indicative of ovarian cancer or the propensity to develop ovarian cancer. In particular embodiments, a 2, 3, 4, 5, or 6-fold change in the level of a marker of the invention is indicative of ovarian cancer. In yet another embodiment, an expression profile that characterizes alterations in the expression two or more markers is correlated with a particular disease state (e.g., ovarian cancer). Such correlations are indicative of ovarian cancer or the propensity to develop ovarian cancer. In one embodiment, an ovarian cancer can be monitored using the methods and compositions of the invention.

In one embodiment, the level of one or more markers is measured on at least two different occasions and an alteration in the levels as compared to normal reference levels over time is used as an indicator of ovarian cancer or the propensity to develop ovarian cancer. The level of marker in the biological sample (e.g., cell samples, biopsy sample, blood, blood serum, plasma, saliva, urine, peritoneal fluid, ascites, pleural effusions, and ovarian cyst fluid) of a subject having ovarian cancer or the propensity to develop such a condition may be altered by as little as 10%, 20%, 30%, or 40%, or by as much as 50%, 60%, 70%, 80%, or 90% or more relative to the level of such marker in a normal control. In general, levels of MMP9, tPA, IGFBP2, MMP7, Tenascin, NAP2, Glycodelin, MCSF, MMP2, InhibinA, uPAR, EGFR, Transthyretin (TT or prealbumin), Apolipoprotein A-1 (Apo A-1), beta 2-Microglobulin (beta 2M), Transferrin (Tfr). Cancer Antigen 125 (CA 125 II) and/or HE4 are compared to levels of these markers in a healthy subject (i.e., those who do not have and/or who will not develop ovarian cancer).

Detection methods may include use of a biochip array. Biochip arrays useful in the invention include protein and polynucleotide arrays. One or more markers are captured on the biochip array and subjected to analysis to detect the level of the markers in a sample.

Markers may be captured with capture reagents immobilized to a solid support, such as a biochip, a multiwell microtiter plate, a resin, a nitrocellulose membrane, or on beads that are subsequently probed for the presence or level of a marker. Capture can be on a chromatographic surface or a biospecific surface. For example, a sample containing the markers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant minor method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Mass spectrometry (MS) is a well-known tool for analyzing chemical compounds. Thus, in one embodiment, the methods of the present invention comprise performing quantitative MS to measure the serum peptide marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

The protein fragments, whether they are peptides derived from the main chain of the protein or are residues of a side-chain, are collected on the collection layer. They may then be analyzed by a spectroscopic method based on matrix-assisted laser desorption/ionization (MALDI) or electrospray ionization (ESI). The preferred procedure is MALDI with time of flight (TOF) analysis, known as MALDI-TOF MS.

This involves forming a matrix on the membrane, e.g. as described in the literature, with an agent which absorbs the incident light strongly at the particular wavelength employed. The sample is excited by UV, or IR laser light into the vapour phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are commercially available from PerSeptive Biosystems, Inc. (Framingham, Mass., USA) and are described in the literature, e.g. M. Kussmann and P. Roepstorff, cited above.

Magnetic-based serum processing can be combined with traditional MALDI-TOF. Through this approach, improved peptide capture is achieved prior to matrix mixture and deposition of the sample on MALDI target plates. Accordingly, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on the collecting membrane, and the array may be analyzed. Subsequently, automated output of the results is provided by using the ExPASy server, as at present used for MIDI-TOF MS and to generate the data in a form suitable for computers.

Other techniques for improving the mass accuracy and sensitivity of the MALDI-TOF MS can be used to analyze the fragments of protein obtained on the collection membrane. These include the use of delayed ion extraction, energy reflectors and ion-trap modules. In addition, post source decay and MS--MS analysis are useful to provide further structural analysis. With ESI, the sample is in the liquid phase and the analysis can be by ion-trap, TOF, single quadrupole or multi-quadrupole mass spectrometers. The use of such devices (other than a single quadrupole) allows MS-MS or MSⁿ analysis to be performed. Tandem mass spectrometry allows multiple reactions to be monitored at the same time.

Capillary infusion may be employed to introduce the marker to a desired MS implementation, for instance, because it can efficiently introduce small quantities of a sample into a mass spectrometer without destroying the vacuum. Capillary columns are routinely used to interface the ionization source of a MS with other separation techniques including gas chromatography (GC) and liquid chromatography (LC). GC and LC can serve to separate a solution into its different components prior to mass analysis. Such techniques are readily combined with MS, for instance. One variation of the technique is that high performance liquid chromatography (HPLC) can now be directly coupled to mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem MS experiments. FTMS is based on the principle of a charged particle orbiting in the presence of a magnetic field. Coupled to ESI and MALDI, FTMS offers high accuracy with errors as low as 0.001%.

In one embodiment, the methods of the invention further comprise identifying significant peaks from combined spectra. The methods may also further comprise searching for outlier spectra. In another embodiment, the method of the invention further comprises determining distant dependent K-nearest neighbors.

In another embodiment of the method of the invention, an ion mobility spectrometer can be used to detect and characterize serum peptide markers. The principle of ion mobility spectrometry is based on different mobility of ions.

Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in a sample. One advantage of ion mobility spectrometry is that it can operate at atmospheric pressure.

The diagnostic methods described herein can be used individually or in combination with any other diagnostic method described herein for a more accurate diagnosis of the presence or severity of ovarian cancer.

The diagnostic methods described herein can also be used to monitor and manage ovarian cancer, or to reliably distinguish ovarian cancer from a benign pelvic mass.

As indicated above, the invention provides methods for aiding a human cancer diagnosis using one or more markers, as specified herein. These markers can be used alone, in combination with other markers in any set, or with entirely different markers in aiding human cancer diagnosis. The markers are differentially present in samples of a human cancer patient and a normal subject in whom human cancer is undetectable. Therefore, detection of one or more of these markers in a person would provide useful information regarding the probability that the person may have ovarian cancer or regarding the aggressiveness of the cancer.

The detection of the marker is then correlated with a probable diagnosis of cancer. In some embodiments, the detection of an alteration in the level of a marker (e.g., MMP9, tPA, IGFBP2, MMP7, Tenascin, NAP2, Glycodelin, MCSF, MMP2,

InhibinA, uPAR, EGFR, Transthyretin (TT or prealbumin), Apolipoprotein A-1 (Apo A-1), beta 2-Microglobulin (beta 2M), Transferrin (Tfr), Cancer Antigen 125 (CA 125 II) and/or HE4), without quantifying the amount thereof, is useful and can be correlated with a probable diagnosis of cancer. The measurement of markers may also involve quantifying the markers to correlate the detection of markers with a probable diagnosis of cancer. Thus, if the amount of the markers detected in a subject being tested is different compared to a control amount (i.e., higher or lower than the control), then the subject being tested has a higher probability of having cancer.

The correlation may take into account the amount of the marker or markers in the sample compared to a control amount of the marker or markers (e.g., in normal subjects or in non-cancer subjects, such as where cancer is undetectable). A control can be, e.g., the average or median amount of marker present in comparable samples of normal subjects in normal subjects or in non-cancer subjects such as where cancer is undetectable. The control amount is measured under the same or substantially similar experimental conditions as in measuring the test amount. As a result, the control can be employed as a reference standard, where the normal (non-cancer) phenotype is known, and each result can be compared to that standard, rather than re-running a control.

Accordingly, a marker profile may be obtained from a subject sample and compared to a reference marker profile obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate determination of cancer status.

In certain embodiments of the methods of qualifying cancer status, the methods further comprise managing subject treatment based on the status. The invention also provides for such methods where the markers (or specific combination of markers) are measured again after subject management. In these cases, the methods are used to monitor the status of the cancer, e.g., response to cancer treatment, remission of the disease or progression of the disease.

Any marker, individually, is useful in aiding in the determination of cancer status. First, the selected marker is detected in a subject sample using the methods described herein. Then, the result is compared with a control that distinguishes cancer status from non-cancer status. As is well understood in the art, the techniques can be adjusted to increase sensitivity or specificity of the diagnostic assay depending on the preference of the diagnostician.

While individual markers are useful diagnostic markers, in some instances, a combination of markers provides greater predictive value than single markers alone.

The detection of a plurality of markers (or absence thereof, as the case may be) in a sample can increase the percentage of true positive and true negative diagnoses and decrease the percentage of false positive or false negative diagnoses. Thus, preferred methods of the present invention comprise the measurement of more than one marker.

### Microarrays

As reported herein, a number of markers (e.g., MMP9, tPA, IGFBP2, MMP7, Tenascin, NAP2, Glycodelin, MCSF, MMP2, InhibinA, uPAR, EGFR, Transthyretin (TT or prealbumin), Apolipoprotein A-1 (Apo A-1), beta 2-Microglobulin (beta 2M), Transferrin (Tfr), Cancer Antigen 125 (CA 125 II) and/or HE4) have been identified that are associated with ovarian cancer. Methods for assaying the expression of these polypeptides are useful for characterizing ovarian cancer. In particular, the invention provides diagnostic methods and compositions useful for identifying a polypeptide expression profile that identifies a subject as having or having a propensity to develop ovarian cancer. Such assays can be used to measure an alteration in the level of a polypeptide.

The polypeptides and nucleic acid molecules of the invention are useful as hybridizable array elements in a microarray. The array elements are organized in an ordered fashion such that each element is present at a specified location on the substrate. Useful substrate materials include beads, membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al.(Nature Genet. 26:283-289), and in U.S. Pat. No. 6,436,665, hereby incorporated by reference.

### Protein Microarrays

Proteins (e.g., MMP9, tPA, IGFBP2, MMP7, Tenascin, NAP2, Glycodelin, MCSF, MMP2, InhibinA, uPAR, EGFR, Transthyretin (TT or prealbumin), Apolipoprotein A-1 (Apo A-1), beta 2-Microglobulin (beta 2M), Transferrin Cancer Antigen 125 (CA 125 II) and/or HE4) may be analyzed using protein microarrays. Such arrays are useful in high-throughput low-cost screens to identify alterations in the expression or post-translation modification of a polypeptide of the invention, or a fragment thereof. In particular, such microarrays are useful to identify a protein whose expression is altered in ovarian cancer. In one embodiment, a protein microarray of the invention binds a marker present in a subject sample and detects an alteration in the level of the marker. Typically, a protein microarray features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

The protein microarray is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine); a homogenized tissue sample (e.g. a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

### Nucleic Acid Microarrays

To produce a nucleic acid microarray, oligonucleotides derived from a MMP9, tPA, IGFBP2, MMP7, Tenascin, NAP2, Glycodelin, MCSF, MMP2, InhibinA, uPAR, EGFR, Transthyretin (TT or prealbumin), Apolipoprotein A-1 (Apo A-1), beta 2-Microglobulin (beta 2M), Transferrin (Tfr), Cancer Antigen 125 (CA 125 II) and/or HE4 nucleic acid molecule may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, preferably as a bodily fluid (such as blood, blood serum, plasma, saliva, urine, peritoneal fluid, ovarian cyst fluid) or tissue sample (e.g. a tissue sample obtained by biopsy). For some applications, cultured cells or other tissue preparations may be used. The mRNA is isolated according to standard methods, and cDNA is produced and used as a template to make complementary RNA suitable for hybridization. Such methods are known in the art. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30 C., more preferably of at least about 37 C., and most preferably of at least about 42 C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30 C in 750 mM NaCl, 75 mM trisodium citrate, and 1% SDS. In a more preferred embodiment, hybridization will occur at 37 C. in 500 mM NaCl, 50 mM trisodium citrate, 1% SDS, 35% formamide, and 100 μg/ml denatured salmon sperm DNA (ssDNA). In a most preferred embodiment, hybridization will occur at 42 C in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25 C., more preferably of at least about 42.degree. C., and most preferably of at least about 68 C. In a preferred embodiment, wash steps will occur at 25 C in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a most preferred embodiment, wash steps will occur at 68 C in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct nucleic acid sequences simultaneously (e.g., Heller et al., Proc. Natl. Acad. Sci. 94:2150-2155, 1997). Preferably, a scanner is used to determine the levels and patterns of fluorescence.

### Diagnostic Kits

The invention provides kits for diagnosing or monitoring ovarian cancer, for distinguishing ovarian cancer from a benign pelvic mass, or for monitoring ovarian cancer. In one embodiment, the kit includes a composition containing at least one agent that binds a polypeptide or polynucleotide whose expression is altered in ovarian cancer tissue samples (e.g., cell samples, biopsy samples) and bodily fluids, including, but not limited to, blood, blood serum, plasma, saliva, urine, peritoneal fluid and ovarian cyst fluid. In another embodiment, the invention provides a kit that contains an agent that binds a nucleic acid molecule whose expression is altered in ovarian cancer. In some embodiments, the kit comprises a sterile container which contains the binding agent; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired the kit is provided together with instructions for using the kit to diagnose ovarian cancer. The instructions will generally include information about the use of the composition for diagnosing a subject as having ovarian cancer or having a propensity to develop ovarian cancer. In other embodiments, the instructions include at least one of the following: description of the binding agent; warnings; indications; counter-indications; animal study data; clinical study data; and/or references. The instructions may be printed directly on the container (when present), or as a label applied to the container, or as a separate sheet, pamphlet, card, or folder supplied in or with the container.

### Subject Monitoring

The disease state or treatment of a subject having benign pelvic mass, ovarian cancer, or a propensity to develop such a condition can be monitored using the methods and compositions of the invention. In one embodiment, the expression of markers present in a bodily fluid, such as blood, blood serum, plasma, saliva, urine, peritoneal fluid or ovarian cyst fluid, is monitored. Such monitoring may be useful, for example, in assessing the efficacy of a particular drug in a subject or in assessing disease progression. Therapeutics that decrease the expression of a marker of the invention (e.g., IL-6, MMP9, tPA, IGFBP2, MMP7, Tenascin, NAP2, Glycodelin, MCSF, MMP2, InhibinA, uPAR, EGFR, Transthyretin (TT or prealbumin), Apolipoprotein A-1 (Apo A-1), beta 2-Microglobulin (beta 2M), Transferrin (Tfr), Cancer Antigen 125 (CA 125 II) and/or HE4) are taken as particularly useful in the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Identification of a Panel of Biomarkers that Detect Ovarian Cancer

A panel of biomarkers was identified that provides a high level of specificity among women with benign pelvic masses while maintaining a high level of sensitivity in detection ovarian cancer. This panel of biomarkers includes: MMP9, tPA, IGFBP2, MMP7, Tenascin, NAP2, Glycodelin, MCSF, MMP2, InhibinA, uPAR, EGFR. Some of the biomarkers have been previously reported as associated with ovarian cancer. The current invention provides for the use of such markers not only for the detection of ovarian cancer, but also in distinguishing benign pelvic masses from ovarian cancer. In particular, the use of these biomarkers complements the use of CA125 to enhance the specificity of preoperative assessment of ovarian tumors as likely to be benign or malignant.

ELISA tests of biomarkers were performed on 15 ovarian cancer patients and 22 patients with benign pelvic masses. The biomarkers, MMP7, Tenascin C, NAP2, uPAR, and MMP9, were selected for analysis. The 22 benign patients were identified as having relatively high serum CA125 levels (mean=155.5 IU, median=101.6 IU). For statistical analysis, the biomarkers were first evaluated individually by receiver-operating-characteristic (ROC) curve analysis. The biomarkers, after log transformation, were further assessed by multivariate logistic regression for their significance in complementing CA125 to differentiate malignant from benign pelvic masses.

The area-under-curve (AUC) for CA125 alone was 0.824. The individual AUCs for MMP7, Tenascin C, NAP2, uPAR, and MMP9, were 0.870, 0.685, 0.748, 0.718, and 0 670, respectively. The p-values from logistic regressions testing the significance of each of the five markers in complementing CA125 to detect ovarian cancer from benign pelvic masses were 0.008, 0.052, 0.080, 0.357, 0.212, respectively. Finally, in a multivariate logistic regression model that included CA125, MMP7, Tenascin C, and NAP2 as its input variables, the p-values for the four input variables were 0.105, 0.044, 0.034, and 0.050, respectively.

In a first set of experiments, MMP7, Tenascin C, and NAP2 demonstrated both a relatively high discriminatory power individually and were capable of complementing CA125.

### Example 2

### A Panel of Markers Useful in Distinguishing Malignant from Benign Ovarian Tumors

In other experiments, the following biomarkers: tPA, IGFBP2, MMP2, MMP7, MMP9, MCSF, Inhibin A, Glycodelin, Tenascin C, NAP2, uPAR, and EGFR were identified as having high specificity in preoperative assessment of ovarian tumor for risk of cancer among women with elevated CA125.

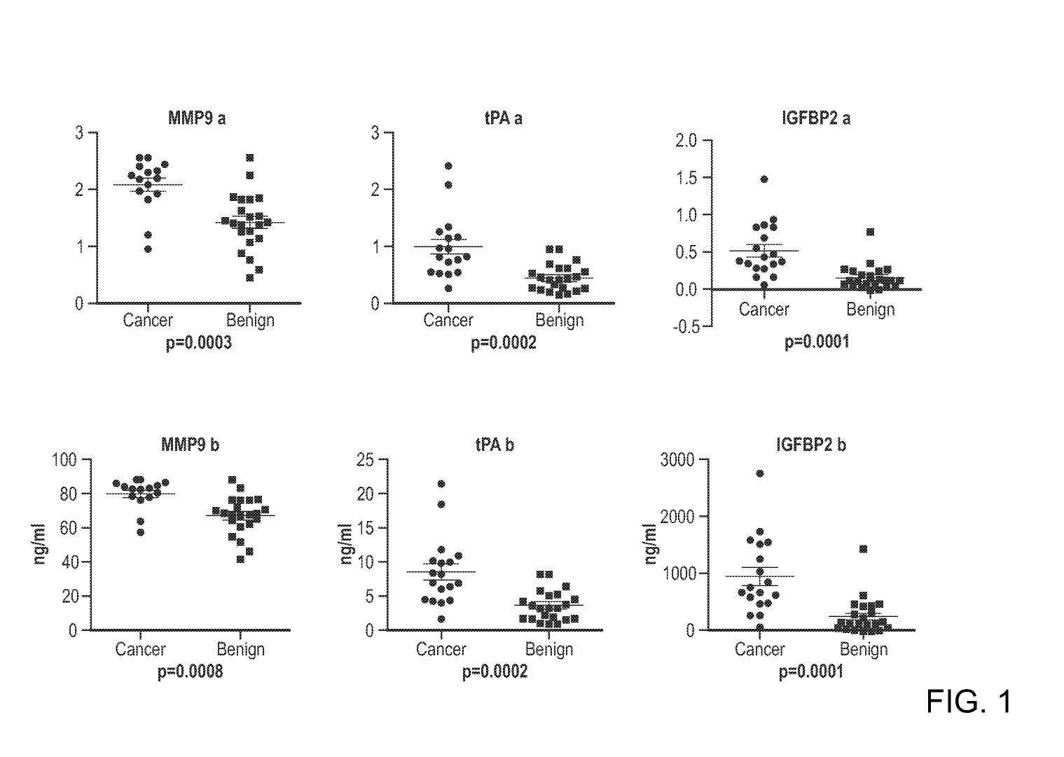

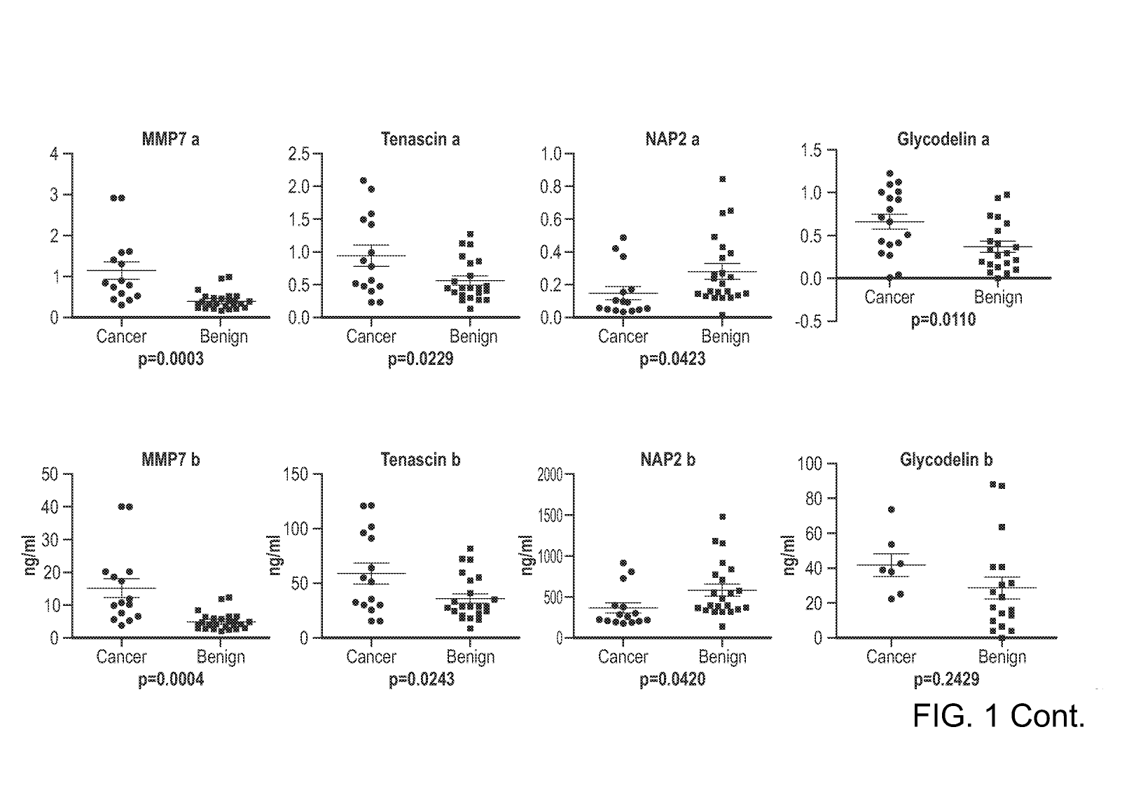

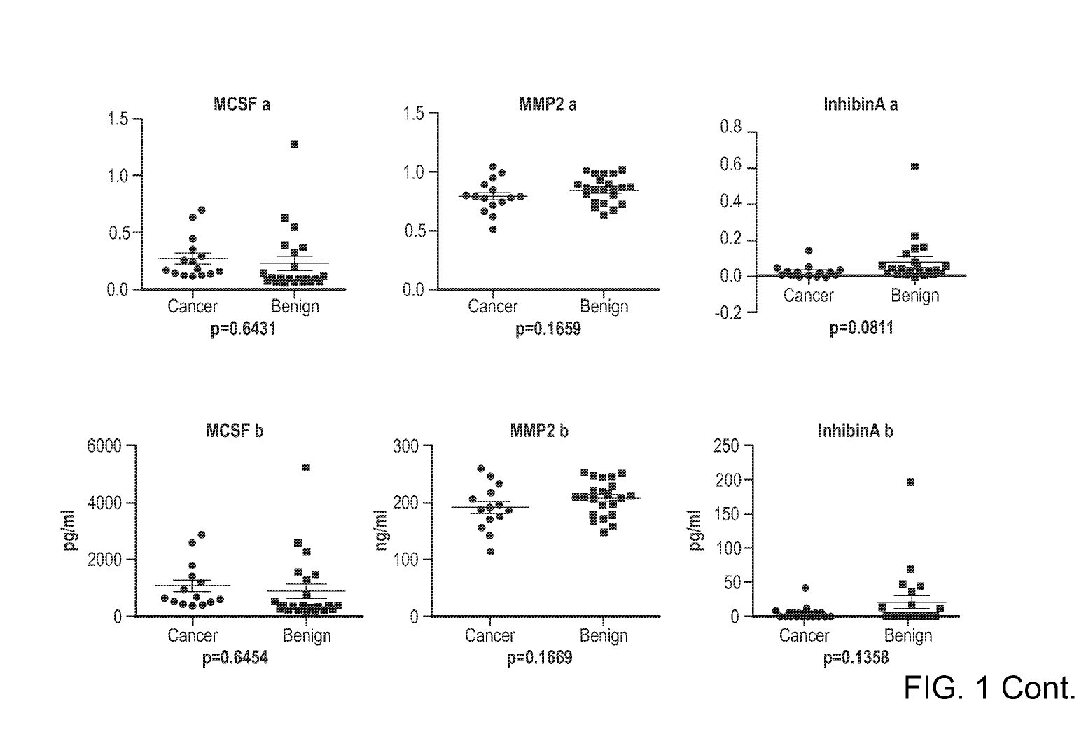

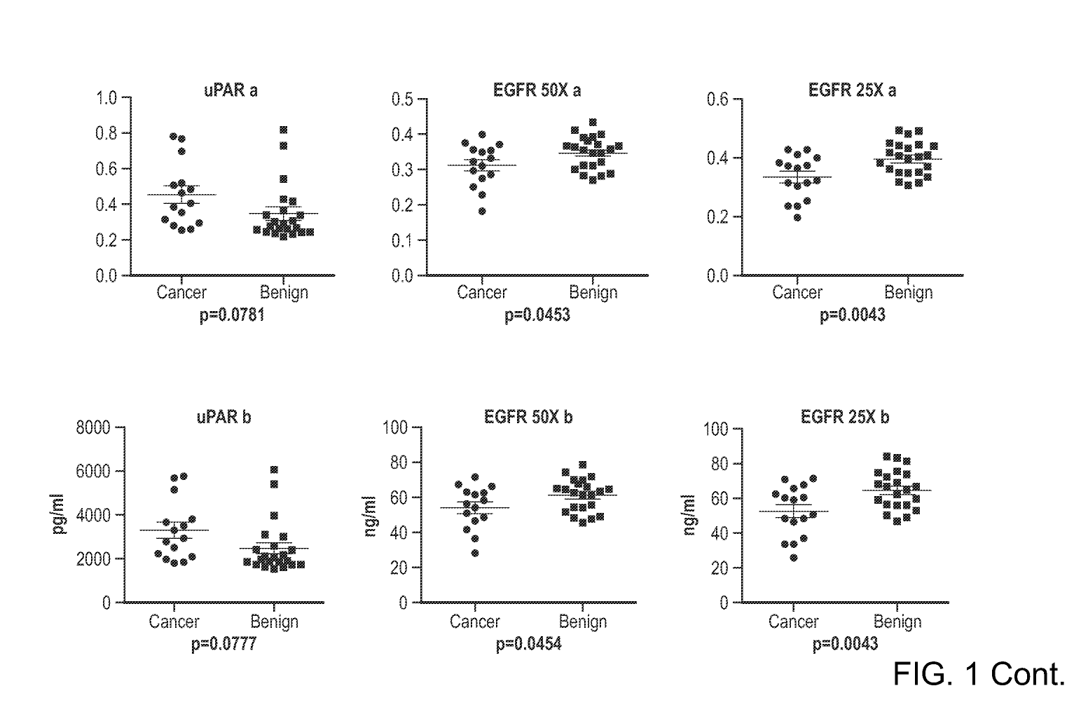

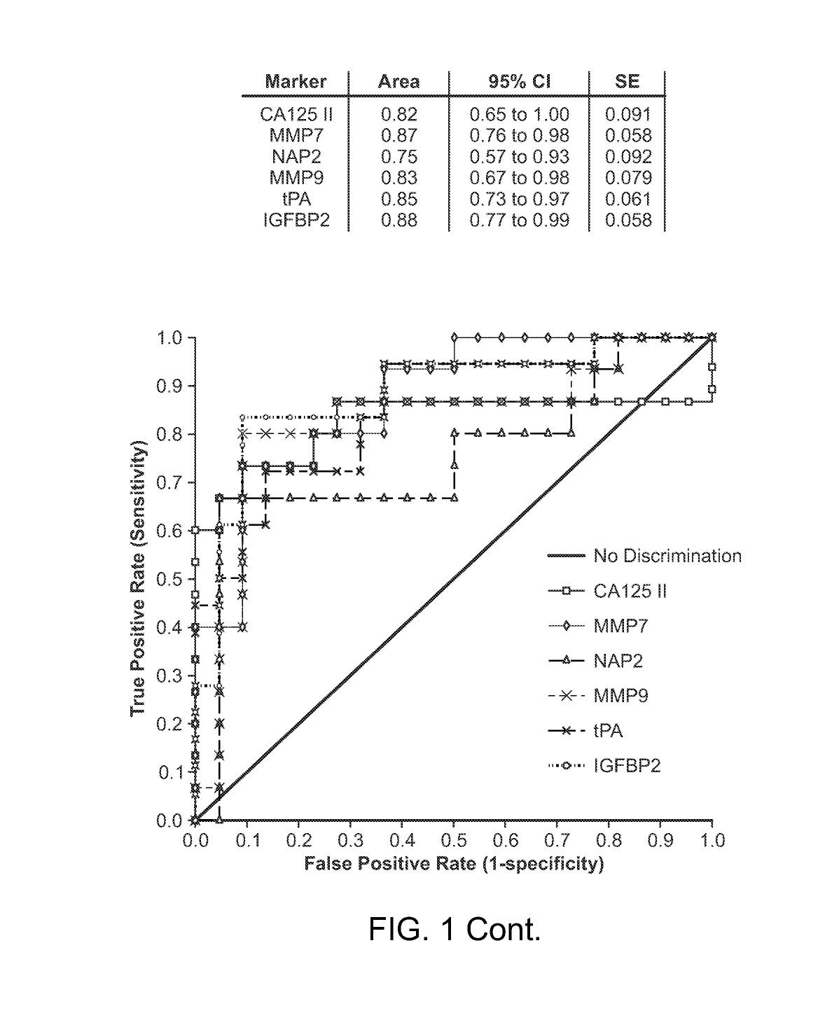

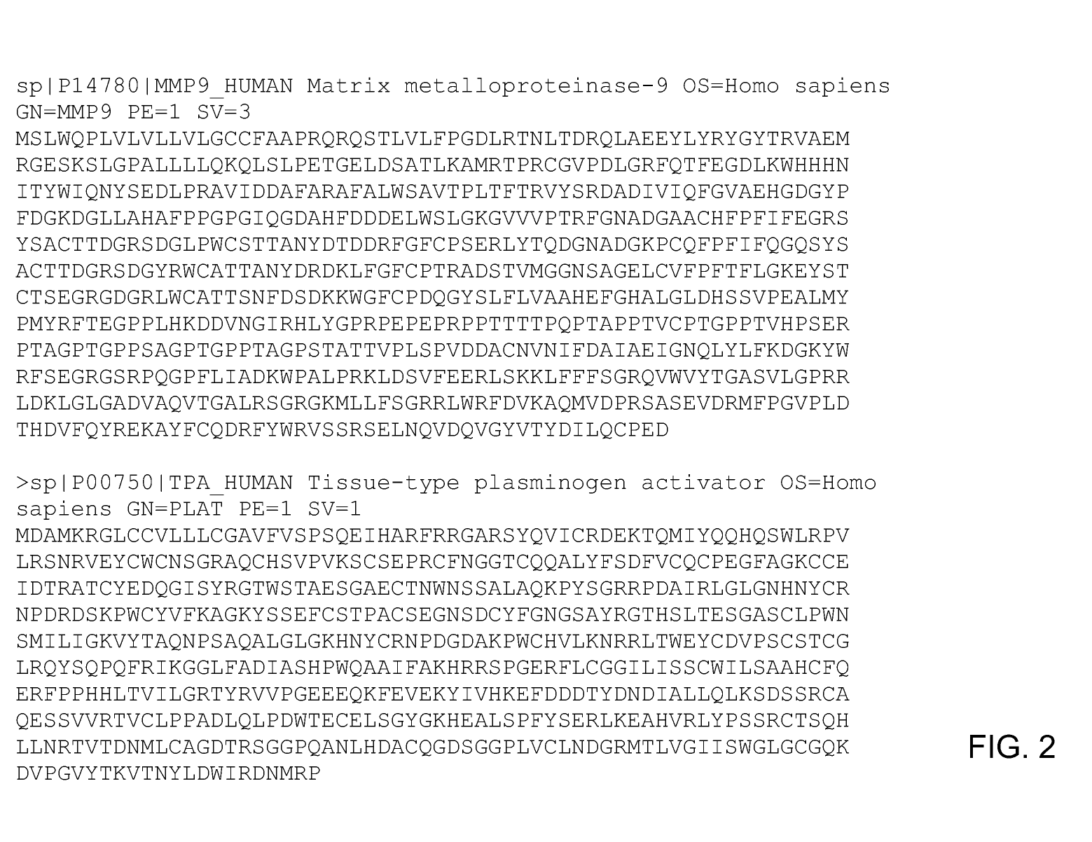

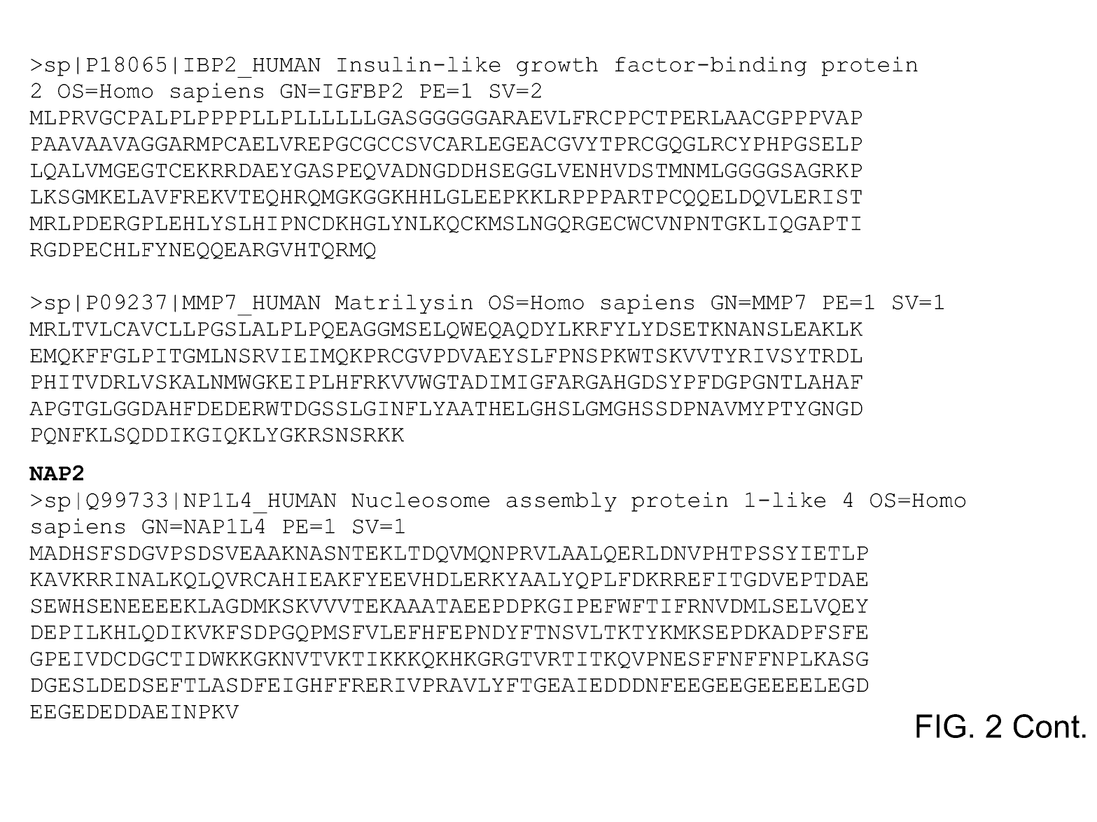

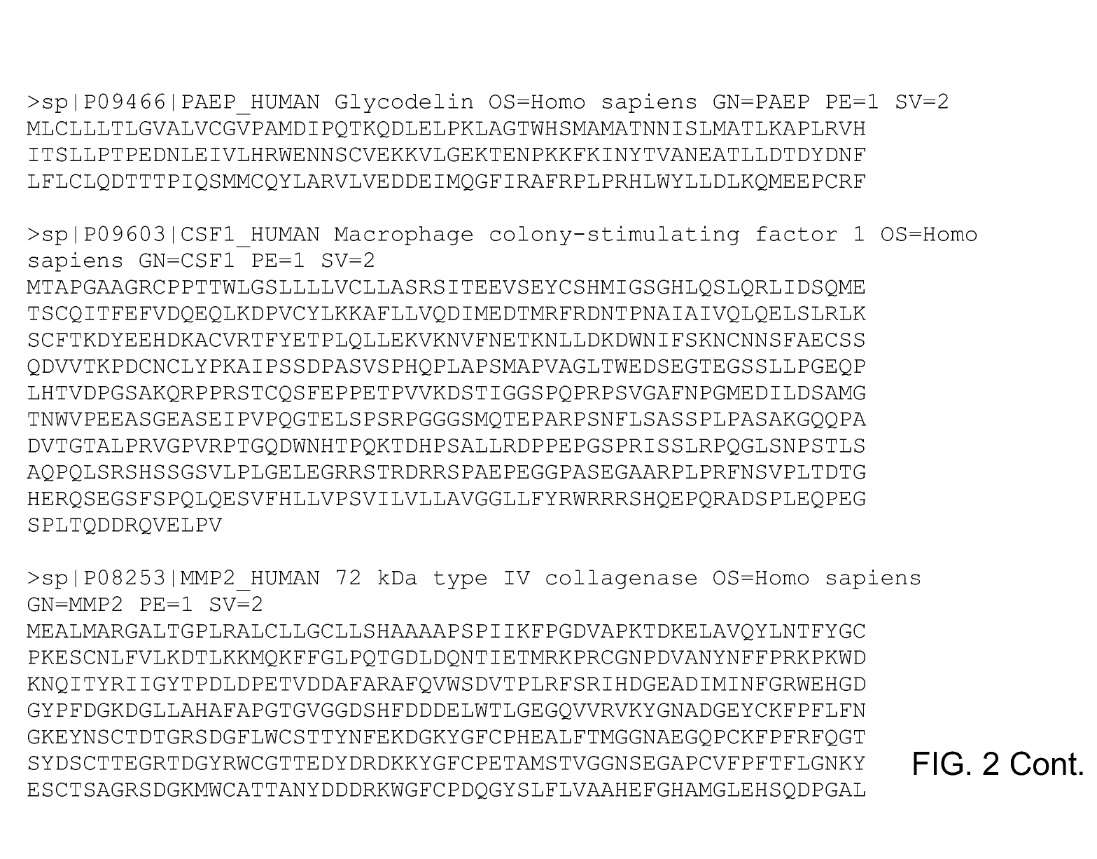

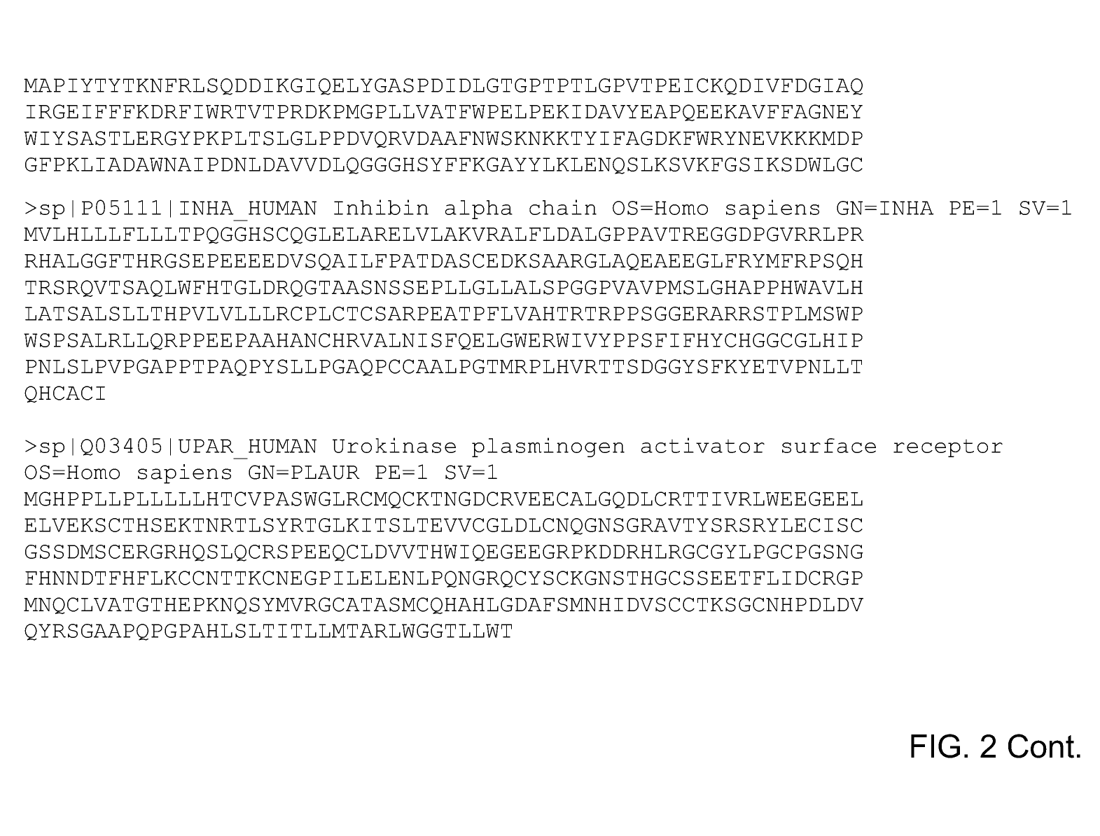

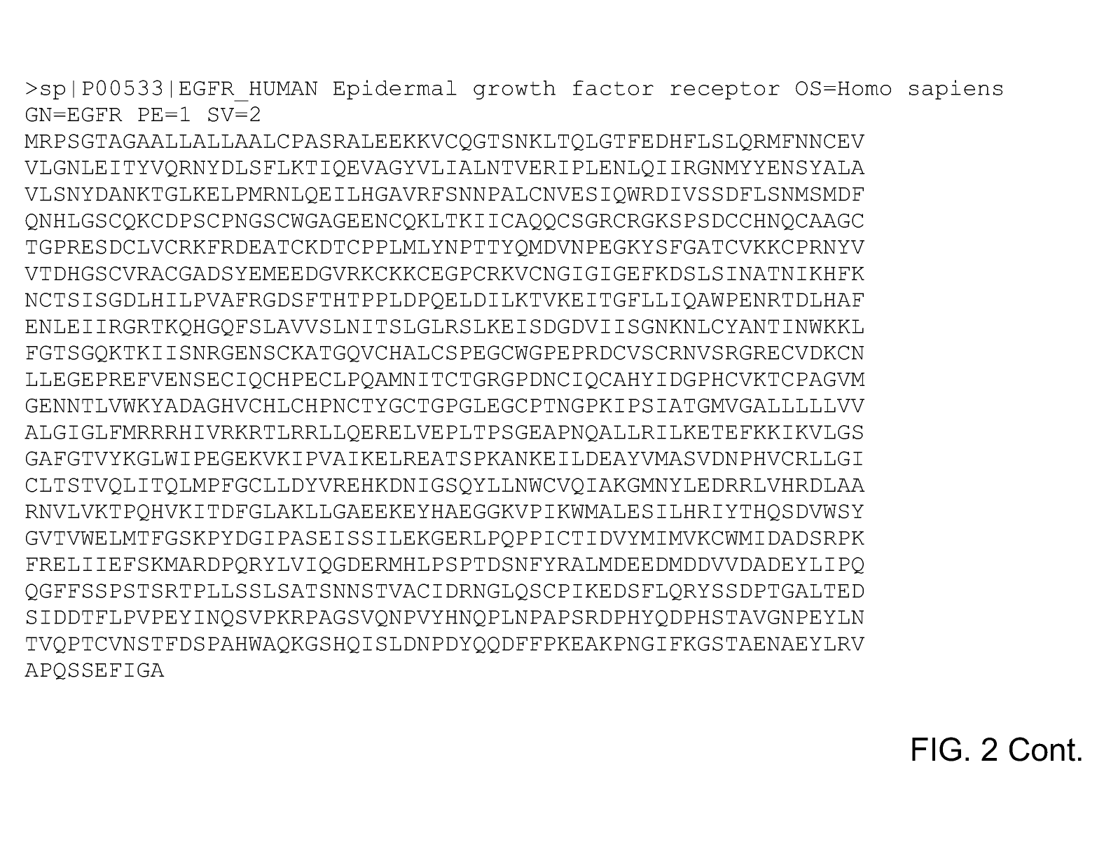

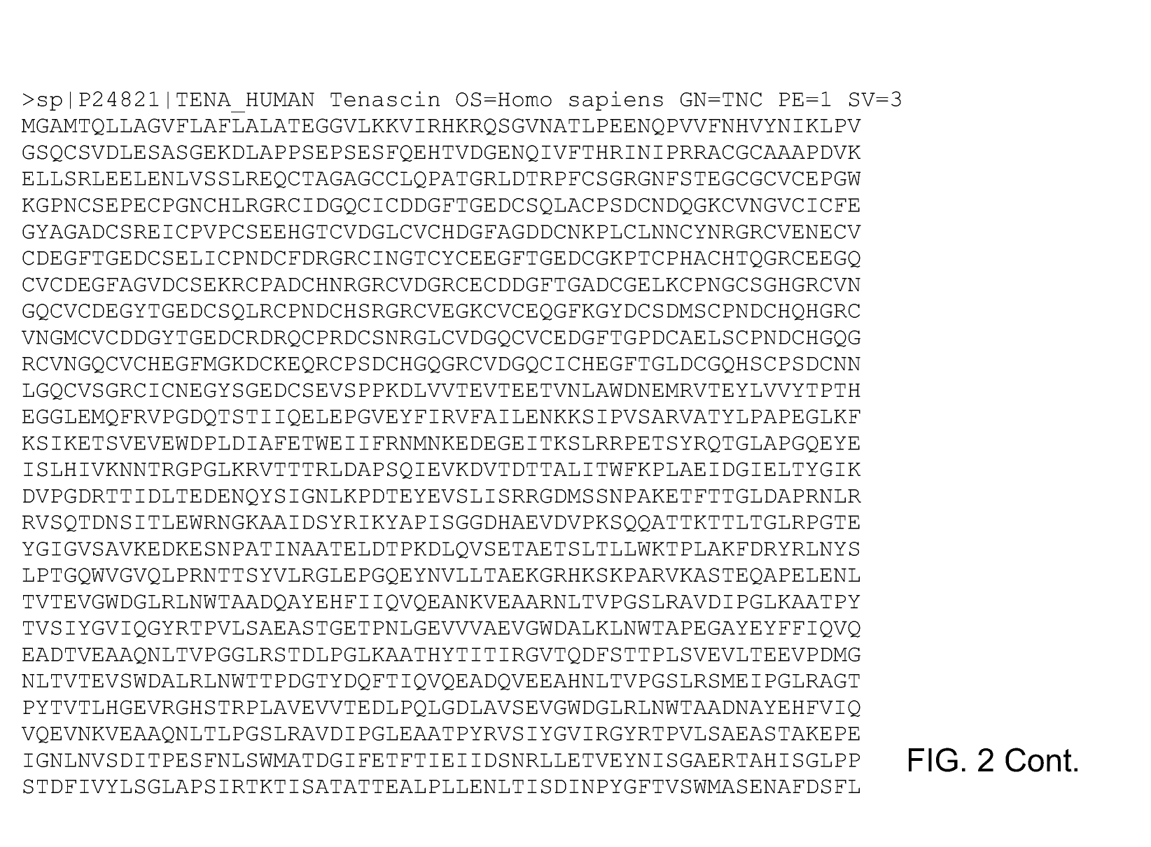

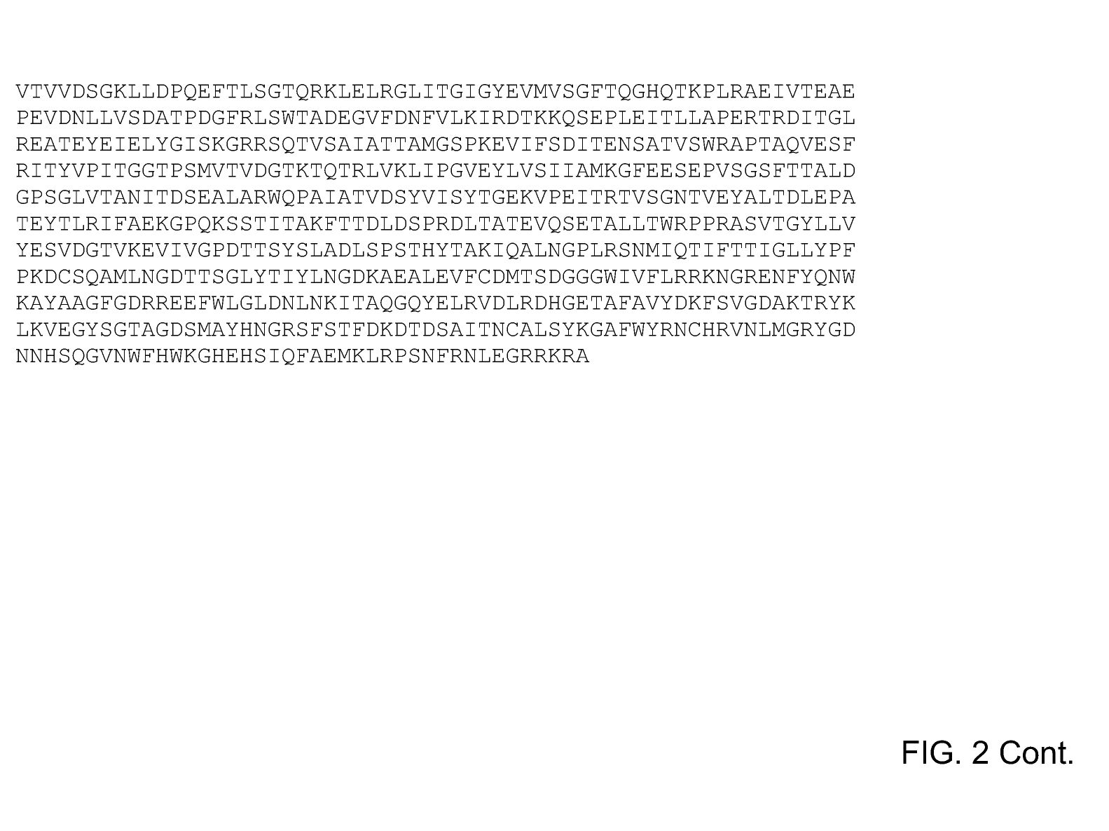

ELISA tests of 12 biomarkers were carried out on tPA, IGFBP2, MMP2, MMP7, MMP9, MCSF, Inhibin A, Glycodelin, Tenascin C, NAP2, uPAR, and EGFR. These biomarkers were selected based on their individual relevancy to ovarian cancer and their ability to be assayed by ELISA. ELISA analyses were performed on 15 ovarian cancer patients and 22 patients with benign pelvic masses and relatively high serum CA125 levels (mean=155.5 IU, median=101.6 IU). The biomarkers were first evaluated individually by ROC curve analysis. The selected biomarkers were further assessed by multivariate logistic regression for their significance in complementing CA125 to differentiate malignant from benign pelvic masses (FIG. 1). Sequences of the analysed biomarkers are shown in FIG. 2.

The area-under-curve (AUC) for CA125 alone w as 0.82. The five markers with the highest AUCs w ere IGFBP2 (0.88), MMP7 (0.87), tPA (0.85), MMP9 (0.83), and NAP2 (0.75). Logistic regression analysis showed that other than tPA, the remaining four markers complemented CA125 with varying yet statistically significant contributions to the separation of cancer from benign. A final multivariate logistic regression model that combined CA125, MMP7, NAP2, and IGFBP2 w ere able to reach an ROC/AUC of 0.98.

In this evaluation of biomarkers, MMP7, MMP9, NAP2, and IGFBP2 demonstrated high discriminatory powers individually and the ability to complement CA125. These biomarkers are useful in a multivariate panel to identify malignant from benign ovarian tumor preoperatively. In further experiments, MMP9, tPA, IGFBP2, MMP7, Tenascin, NAP2, Glycodelin, MCSF, MMP2, InhibinA, uPAR, EGFR were identified as a high level of specificity in distinguishing among women with benign pelvic masses while retaining superior sensitivity in detection ovarian cancer.

These biomarkers are particularly useful as part of a multivariate panel to identify malignant from benign ovarian tumor preoperatively. The biomarkers can be combined with other ovarian cancer biomarkers, such as Transthyretin (TT or prealbumin), Apolipoprotein A-1 (Apo A-1), beta 2-Microglobulin (beta 2M), Transferrin (Tfr) and Cancer Antigen 125 (CA 125 II) and/or HE4. In particular embodiments, MMP9, tPA, IGFBP2, MMP7, Tenascin, NAP2, Glycodelin, MCSF, MMP2, InhibinA, uPAR, EGFR are used in in vitro diagnostic multivariate Index assays (IVDMIA), such as OVA1, as clinical tests for diagnosis or risk assessment to improve specificity in detecting ovarian cancer. Improved diagnostic specificity (reduced false positive rate) will result in better positive predictive value.

### Example 3

### IGFBP2 and MMP7 Distinguished Malignant from Benign Ovarian Tumors

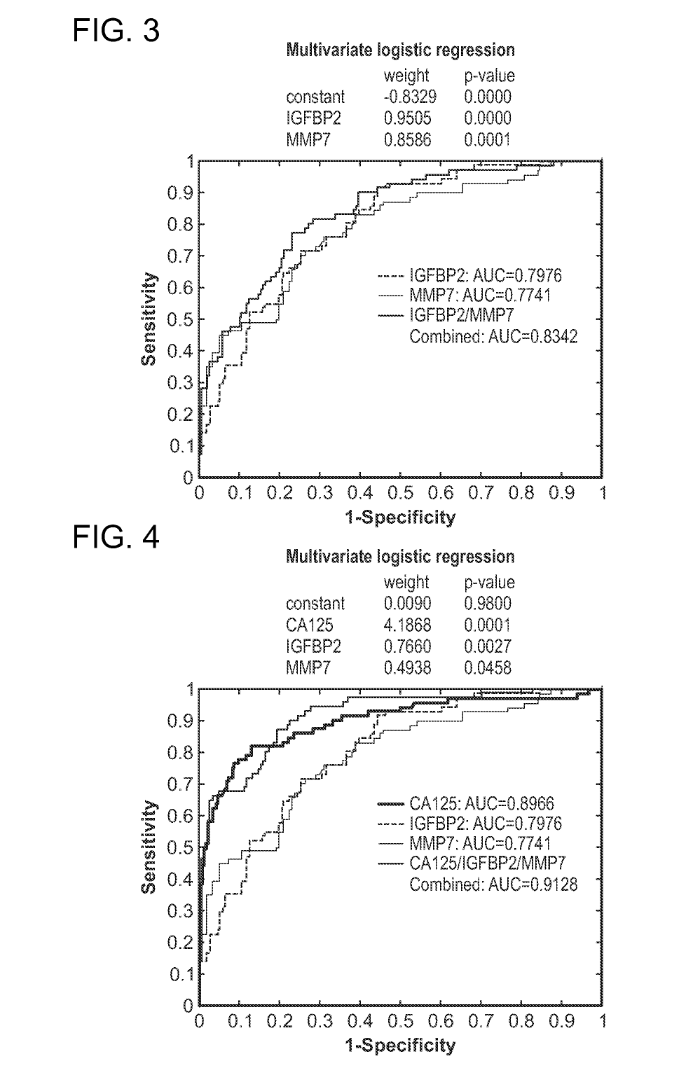

In additional studies using the techniques described above, area-under-curve (AUC) from receiver operating characteristic (ROC) curve analysis demonstrated the discriminatory power of biomarkers individually and in combination in separating malignant from benign ovarian tumor on independent validation samples (n=222). In FIG. 3, IGFBP2 (Insulin-like growth factor-binding protein 2) is shown using a blue dot/dash line, AUC=0.7976. MMP7 (matrix metalloproteinase-7) is shown using a green dash line, AUC=0.7741. The two biomarkers are complementary as shown by ROC of a combination of the two markers through logistic regression, red solid line, AUC=0.8342.

### Example 4

### CA125, IGFBP2, and MMP7 Distinguished Malignant from Benign Ovarian Tumors

In additional studies using the techniques described above, area-under-curve (AUC) from receiver operating characteristic (ROC) curve analysis demonstrated the discriminatory power of biomarkers individually and in combination in separating malignant from benign ovarian tumor on independent validation samples (n=222). In FIG. 4, CA125 is shown using a magenta long dash line, AUC=0.8966. IGFBP2 (Insulin-like growth factor-binding protein 2) is shown using a blue dot/dash line, AUC=0.7976. MMP7 (matrix metalloproteinase-7) is shown using a green dash line, AUC=0.7741. The 3 biomarkers are complementary as shown by ROC of a combination of the 3markers through logistic regression, red solid line, AUC=0.9128.

### Example 5

### IGFBP2 Complements OVA1 in Distinguishing Malignant from Benign Ovarian Tumors

In additional studies using the techniques described above, IGFBP2 was analysed for its ability to complement the commercially available OVA1 test. Results are shown in a scatterplot of IGFBP2 vs. OVA1 (a single-valued index combining CA125, transferrin, transthyretin (prealbumin), apolipoprotein A1, and beta 2 microglobulin through multivariate analysis), n=222 (FIG. 4). Open red circles show malignant ovarian tumor and green filled circles show benign ovarian tumor. Solid red line indicates possible linear or nonlinear classifiers combining IGFBP2 and OVA1 to improve performance of OVA1.

### Example 6

### HE4 Complements OVA1 in Distinguishing Malignant from Benign Ovarian Tumors

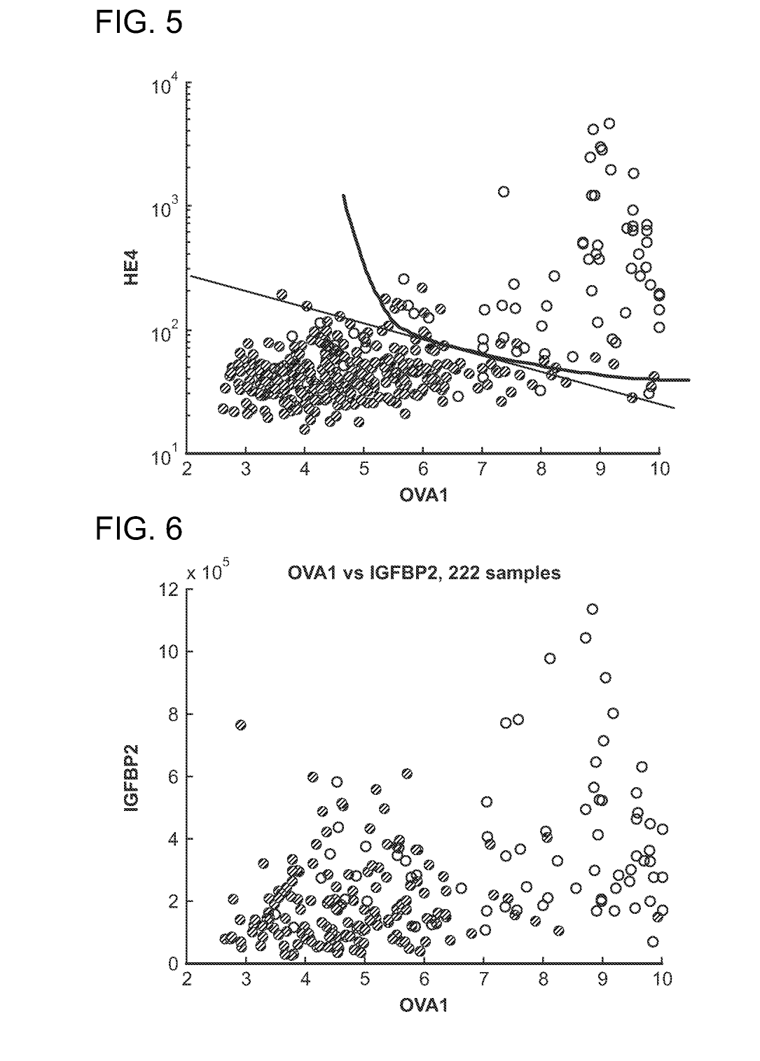

In additional studies using the techniques described above, HE4 was analysed for its ability to complement the commercially available OVA1 test. Results are shown in a scatterplot of HE4 vs. OVA1 (a single-valued index combining CA125, transferrin, transthyretin (prealbumin), apolipoprotein A1, and beta 2 microglobulin through multivariate analysis), n=222 (FIG. 5). Open red circles show malignant ovarian tumor and green filled circles show benign ovarian tumor. The solid red line indicates possible linear or nonlinear classifiers combining HE4 and OVA1 to improve performance of OVA1.

### Example 7

### IGFBP2 Complements OVA1 in Distinguishing Malignant from Benign Ovarian Tumors

In additional studies using the techniques described above, IGFBP2 was analysed for its ability to complement the commercially available OVA1 test(a single-valued index combining CA125, transferrin, transthyretin (prealbumin), apolipoprotein A1, and beta 2 microglobulin through multivariate analysis), n=222 (FIG. 6). Results are illustrated in a scatter plot where open red circles are malignant ovarian tumor and green filled circles are benign ovarian tumor. Solid red line indicate possible linear or nonlinear classifiers combining IGFBP2 and OVA1 to improve performance of OVA1.

### Example 8

### IGFBP2, MMP7, and tPA Distinguished Malignant from Benign Ovarian Tumors in a Bead Based Assay

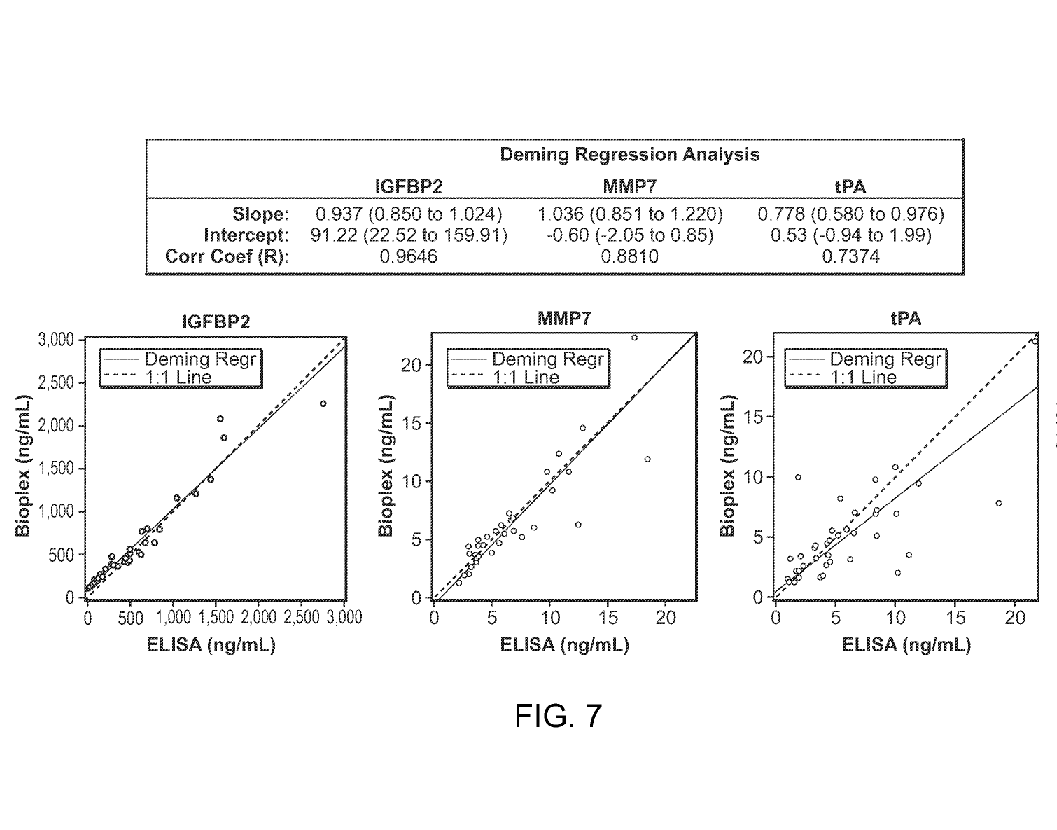

Biomarkers were also measured using beads-based assays on the Luminex/Bioplex platform using samples from patients with malignant and benign ovarian tumors. FIG. 7 shows the method correlation between the Bioplex 200 Assays and the ELISAs of biomarkers IGFBP2, MMP7, and tPA.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. (canceled)

2. A method for identifying ovarian cancer in a subject or distinguishing ovarian cancer from a benign pelvic mass in a subject, the method comprising measuring the level of CA125 and one or more markers selected from the group consisting of MMP9, tPA, IGFBP2, MMP7, Tenascin, NAP2, Glycodelin, MCSF, MMP2, InhibinA, uPAR, and EGFR in a biological sample derived from the subject, wherein an alteration in said level relative to the level present in a reference, identifies ovarian cancer in the subject, and failure to identify an alteration in said levels identifies the subject as having a benign pelvic mass.

3. The method of claim 2, wherein the biological sample is a biological sample selected from the group consisting of ovarian tissue sample, tumor sample, needle biopsy, blood, blood serum, plasma, ascites, pleural effusion, and urine.

4. The method of claim 2, wherein the Marker nucleic acid molecule or polypeptide is selected from the group consisting of MMP7, Tenascin C, NAP2, uPAR, and MMP9.

5. The method of claim 2, wherein the Markers are
(a) MMP7, Tenascin C and NAP2;
(b) IGFBP2 and MMP7
(c) IGFBP2, MMP7 and CA125
(d) IGFBP2, MMP7 and CA125
(e) HE4, Transthyretin, Apolipoprotein A-I, beta 2-Micro globulin, Transferrin and Cancer Antigen 125;
(f) IGFBP2, MMP7, and tPA

6. (canceled)

7. (canceled)

8. (canceled)

9. (canceled)

10. The method of claim 2, wherein the Markers further comprise IL-6.

11. (canceled)

12. A method for identifying ovarian cancer in a subject or distinguishing ovarian cancer from a benign pelvic mass in a subject, the method comprising identifying a subject as having an increased level of a CA125 or HE4 in a biological sample of the subject, and measuring the level of CA125 and one or more Marker nucleic acid molecules or polypeptides selected from the group consisting of MMP9, tPA, IGFBP2, MMP7, Tenascin, NAP2, Glycodelin, MCSF, MMP2, InhibinA, uPAR, and EGFR in a biological sample derived from the subject, wherein an alteration in said Marker level relative to the level present in a reference, identifies ovarian cancer in the subject, and failure to identify an alteration in said levels identifies the subject as having a benign pelvic mass.

13. The method of claim 2, wherein the reference is a corresponding biological sample derived from a healthy subject.

14. A method for determining the Marker profile of ovarian cancer, the method comprising quantifying the level of one or more Markers selected from the group consisting of MMP9, tPA, IGFBP2, MMP7, Tenascin, NAP2, Glycodelin, MCSF, MMP2, InhibinA, uPAR, and EGFR in a biologic sample of the subject, wherein the level of Marker in the sample relative to the level in a reference determines the Marker profile of the ovarian cancer.

15. The method of claim 2, further comprising quantifying the level of Transthyretin (TT or prealbumin), Apolipoprotein A-I (Apo A-I), and/or beta 2-Microglobulin (beta 2M), Transferrin (Tfr) and Cancer Antigen 125 (CA 125 II) and/or HE4 in a biological sample derived from the subject.

16. The method of claim 2, wherein the Marker is measured in an immunoassay, radioassay, hybridization assay, mass spectrometry assay, or a multiplexed assay.

17. The method of claim 11, wherein the immunoassay is an ELISA.

18. A kit for identifying ovarian cancer in a biological sample, the kit comprising at least one polynucleotide molecule or capture molecule capable of specifically binding or hybridizing to a MMP9, tPA, IGFBP2, MMP7, Tenascin, NAP2, Glycodelin, MCSF, MMP2, InhibinA, uPAR, or EGFR polypeptide or nucleic acid molecule, and directions for using the polynucleotide or capture molecule for the diagnosis of ovarian cancer according to the method of claim 2.

19. The kit of claim 18, wherein the capture molecule is an antibody, and antibody binding is detected by fluorescence, by autoradiography, by an immunoassay, by an enzymatic assay, or by a colorimetric assay.

20. A microarray or panel of markers comprising
(a) at least two nucleic acid molecules, or fragments thereof, bound to a solid support, wherein the two nucleic acid molecules hybridize to a nucleic acid molecule selected from the group consisting of MMP9, tPA, IGFBP2, MMP7, Tenascin, NAP2, Glycodelin, MCSF, MMP2, InhibinA, uPAR, and EGFR.
(b) at least two polypeptides, or fragments thereof, bound to a solid support, wherein the two polypeptides are selected from the group consisting of MMP9, tPA, IGFBP2, MMP7, Tenascin, NAP2, Glycodelin, MCSF, MMP2, InhibinA, uPAR, and EGFR
(c) at least two capture molecules, antibodies, or fragments thereof, bound to a solid support, wherein the capture molecules or antibodies specifically bind to two or more polypeptides selected from the group consisting of MMP9, tPA, IGFBP2, MMP7, Tenascin, NAP2, Glycodelin, MCSF, MMP2, InhibinA, uPAR, and EGFR

21. (canceled)

22. (canceled)

23. The microarrays or panels of claim 20, further comprising one or more of the following: Transthyretin, Apo A-I, beta 2-Microglobulin, Transferrin and Cancer Antigen 125 polypeptides, polynucleotides, or antibodies that specifically bind said polypeptides.

24-25. (canceled)
